(19) United States
(12) Patent Application Publication
(10) Pub. No.: US 2016/0100769 A1
(43) Pub. Date: Apr. 14, 2016

(54) DEVICE AND METHOD FOR DENOISING OF ELECTROENCEPHALOGRAPHY SIGNAL USING SEGMENT-BASED PRINCIPAL COMPONENT ANALYSIS

(71) Applicant: KOREA UNIVERSITY RESEARCH AND BUSINESS FOUNDATION, Seoul (KR)

(72) Inventors: Hyun-Chul KIM, Seoul (KR); Jong-Hwan LEE, Seoul (KR)

(73) Assignee: KOREA UNIVERSITY RESEARCH AND BUSINESS FOUNDATION, Seoul (KR)

(21) Appl. No.: 14/657,485

(22) Filed: Mar. 13, 2015

(30) Foreign Application Priority Data
Oct. 14, 2014 (KR) 10-2014-0138087

(51) Int. Cl. A61B 5/04; A61B 5/0476

(52) U.S. Cl. CPC A61B 5/04012; A61B 5/0476

(57) ABSTRACT

Provided is a method for denoising of electroencephalography. The method for denoising of electroencephalography (EEG) includes: generating a two-dimensional data matrix (X) from a one-dimensional EEG signal (x), based on segmentation; generating an eigenvector matrix (E) from the two-dimensional data matrix (X), using principal component analysis (PCA); and removing noise in the one-dimensional EEG signal (x), based on a center-frequency and kurtosis for each of a plurality of eigenvectors. The device for denoising of electroencephalography is also provided.

### CROSS-REFERENCE TO RELATED APPLICATIONS

This application claims priority under 35 U.S.C. § 119(a) to Korean Patent Application No. 10-2014-0138087 filed on Oct. 14, 2014, the disclosure of which is incorporated by reference in its entirety herein.

### BACKGROUND

1. Field

Embodiments according to the present invention generally relate to a device and method for denoising of electroencephalography (EEG) signal and particularly, to a device and method for removing noise in EEG signal, using principal component analysis (PCA).

2. Description of Related Art

There are devices and techniques for non-invasive detection and measurement of brain activity and signal, such as device(s) for fMRI (functional magnetic resonance imaging), EEG, MEG (magnetoencephalography), PET (positron emission tomography), and fNIRS (functional near-infrared spectroscopy).

However, each of the devices has advantages and disadvantages in terms of temporal and spatial resolutions. For example, for the fMRI device, while its spatial resolution is superior, its temporal resolution is low compared to that of other devices. To the contrary, for the EEG device, while its spatial resolution is low compared to that of other devices, its temporal resolution is superior. Thus, multi-modal techniques for detecting brain signal, such as a simultaneous or concurrent fMRI-EEG or fNIRS-EEG techniques, are widely used to supplement the resolution(s) for each of the devices.

In the fMRI-EEG technique or simultaneous detection of EEG and fMRI signals, independent component analysis (ICA) is generally applied to remove helium pump noise or cryogenic pump noise among a plurality of noises in the EEG signal.

The ICA uses EEG signals across all channels to extract (or separate) and remove independent components related to the helium pump noise. However, since the independent components extracted are derived from the signals across all channels, the independent components acquire mixed component properties (i.e., neuronal and non-neuronal noise components) in frequency domain. Accordingly, it is difficult to effectively remove the helium pump noise based on the ICA.

### SUMMARY

According to an embodiment of the present invention, a method for denoising of electroencephalography comprises: generating a two-dimensional data matrix (X) from a one-dimensional EEG signal (x), based on segmentation; generating an eigenvector matrix (E) from the two-dimensional data matrix (X), using PCA; and removing noise in the one-dimensional EEG signal (x), based on a center-frequency and kurtosis for each of a plurality of eigenvectors.

Also, according to an embodiment of the present invention, a device for denoising of EEG signal comprises: a data matrix generation module for generating a two-dimensional data matrix (X) from a one-dimensional EEG signal (x), based on segmentation; a PCA module for generating an eigenvector matrix (E) from the two-dimensional data matrix (X), using PCA; and a noise removal module for removing noise in the one-dimensional EEG signal (x), based on a center-frequency and kurtosis for each of a plurality of eigenvectors.

### BRIEF DESCRIPTION OF DRAWINGS

FIG. 1 shows a device for denoising of EEG signal, according to an embodiment.

FIG. 2 shows a diagram for describing a method of generating a two-dimensional (2-D) data matrix by a data matrix generation module shown in FIG. 1.

FIG. 3 shows a second noise removal module shown in FIG. 1, according to an embodiment.

FIG. 4 shows eigenvectors generated by a data matrix generation module shown in FIG. 1 and a center-frequency for each of eigenvectors determined by a Fourier transformer shown in FIG. 3.

FIG. 5 shows a second noise removal module shown in FIG. 1, according to another embodiment.

FIG. 6 shows an eigenvector having multiple peaks as determined and two eigenvectors as separated by a recursion analyzer shown in FIG. 5.

FIG. 7 shows a flow chart of method for denoising of EEG signal, using the device for denoising of EEG signal shown in FIG. 1.

### DESCRIPTION

Hereinafter, exemplary embodiments of the present invention are described with reference to the accompanying drawings. To note, the present invention is not limited to the exemplary embodiments described or a particular embodiment therein but may be implemented in various different ways. The present invention may be modified and take various other forms, without departing from the spirit and technical scope of the present invention.

Terms used herein are used only to describe specific exemplary embodiments and are not intended to limit the present invention. Terms such as “including” and “having” do not limit the present invention to features, number, step, operation, and parts or elements described; others may exist, be added or modified.

Further, unless otherwise stated, when one element is described, for example, as being “connected” or “coupled” to another element, the elements may be directly linked or indirectly linked (i.e., there may be an intermediate element between the elements). Similar concept applies to terms such as “between” and “adjacent to.” Also, unless otherwise clearly stated, a singular expression includes meaning of plural expressions.

Terms such as “first” and “second” may be used to describe various parts or elements and should also not be limited to a particular part or element. The terms are used to distinguish one element from another element. For example, a first element may be designated as a second element, and vice versa, without departing from the technical scope of the present invention.

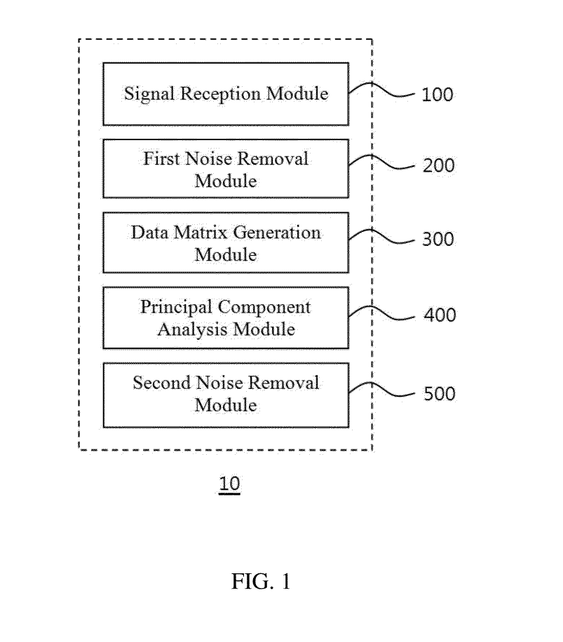

FIG. 1 shows a device for denoising of EEG signal 10, according to an embodiment.

Referring to FIG. 1, the device for denoising of EEG signal 10 comprises a signal reception module 100, a data matrix generation module 300, a PCA module 400, and a second noise removal module 500. According to another embodiment, the device 10 may further comprise a first noise removal module 200, as shown in FIG. 1. The device 10 may receive EEG signal from an EEG detection or measurement device and remove noise in the EEG signal received. According to the embodiment(s), the device 10 may be implemented as a part of the EEG detection or measurement device.

The signal reception module 100 may receive the EEG signal from the EEG measurement device. The EEG signal may be a plurality of signals, with each of the signals from each of multiple channels, or a signal from a single or one particular channel. The EEG signal may be a signal with or without a first noise removed.

The first noise removal module 200 may remove the first noise in the EEG signal received by the signal reception module 100. The first noise may include at least one of magnetic resonance (MR) gradient artifact/noise, electrocardiography noise, and ballistocardiogram noise. The first noise removal module 200 may use conventional technique such as average artifact subtraction (AAS) to remove the first noise.

The data matrix generation module 300 may generate a two-dimensional (2-D) data matrix (X) from the EEG signal with the first noise removed by the first noise removal module 200 or from the EEG signal received by the signal reception module 100. The data matrix generation module 300 may generate the two-dimensional (2-D) data matrix (X) from a one-dimensional EEG signal (x) detected or measured from one channel.

The PCA module 400 may generate eigenvectors or an eigenvector matrix (E) from the two-dimensional (2-D) data matrix (X).

In more detail, the covariance matrix (Cov(X)) generated from the two-dimensional (2-D) data matrix (X) is used as input data for PCA to estimate, extract, or generate eigenvalues or eigenvalue matrix (D) and/or the eigenvector matrix (E), according to Equation (1) below.

Cov(X)=E(XX^{T})=EDE^{T} (1)

The second noise removal module 500 may remove a second noise in the EEG signal. The second noise may be helium pump noise or cryogenic pump noise. The second noise removal module 500 is described in detail later, referring to FIG. 3 and FIG. 4.

The EEG signal measured by the fMRI-EEG technique includes noise of a helium pump or a cryogenic pump operating in an MRI device. In the MRI device, helium performs a function of maintaining superconducting properties of magnet by cryogenically freezing the magnet and helping with a use of the superconducting magnet in the MRI device. Such helium is inbuilt in the MRI device, and constant or continuous operation of the helium pump or the cryogenic pump is required to maintain temperature and humidity of such helium to be constant. This continuously operating helium pump or cryogenic pump has a large impact on the EEG signal acquired during fMRI-EEG measurement and particularly, hinders high-frequency research (higher than 30 Hz). Therefore, an effective noise removal without EEG signal loss is needed.

Each of the elements or components for the device for denoising of EEG signal 10, as shown in FIG. 1, may be functionally and conceptually separable, and persons ordinarily skilled in the art will readily understand that each of the elements may not necessarily be categorized as a separate physical device or be executed by a particular code.

Also, the various modules described may indicate a functional and structural combination or incorporation of hardware and software for driving the hardware, for performing technical concept of the present invention. For example, the modules may be a given code and hardware (resource) for executing the given code and may not necessarily be physically connected code or a particular type of hardware.

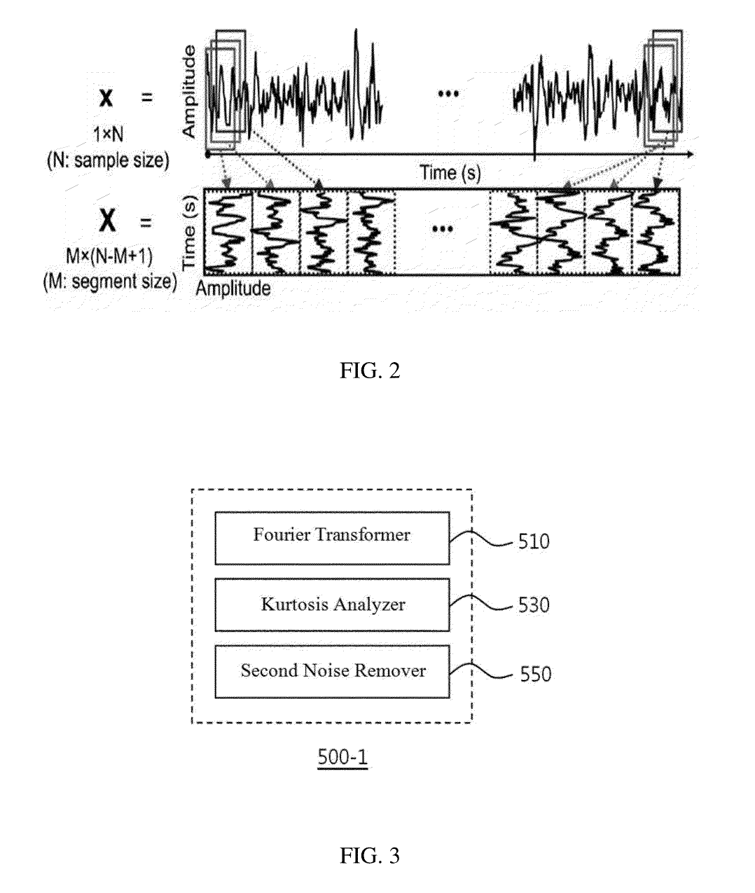

FIG. 2 shows a diagram for describing a method of generating the two-dimensional (2-D) data matrix by the data matrix generation module 300 shown in FIG. 1.

Referring to FIG. 1 and FIG. 2, the data matrix generation module 300 may separate or divide the one-dimensional EEG signal (x) into a plurality of segments and generate a two-dimensional (2-D) data matrix(X) having as column components, data in each of the segments.

In more detail, when a size of the one-dimensional EEG signal (x) is 1×N (where N>1) and a size of each of the segments is M (where 1<M<N), the data matrix generation module 300 may generate a (N-M+1) number of segments while going from a first data point to/through another data point in the one-dimensional EEG signal (x). Therefore, the data matrix generation module 300 may generate a two-dimensional (2-D) data matrix (X) of [M×(N-M+1)] having as component(s), data in each of the (N-M+1) number of segments. That is, the data matrix generation module 300 may generate the two-dimensional (2-D) data matrix (X) having as column component(s), the segments rotated 90 degrees in a clockwise direction or the two-dimensional (2-D) data matrix (X) having as column component(s), 90 degrees in a counter-clockwise direction.

FIG. 3 shows an embodiment of the second noise removal module shown in FIG. 1.

Referring to FIG. 1 and FIG. 3, the second noise removal module 500-1 comprises a Fourier transformer 510, a kurtosis analyzer 530, and a second noise remover 550.

The Fourier transformer 510 may analyze and extract a center-frequency (f_{c}) of the eigenvectors in the eigenvector matrix (E) estimated or generated by the data matrix generation module 300. In more detail, the Fourier transformer 510 may determine the center-frequency (f_{c}) of each eigenvector (eᵢ) by applying Fourier transform or Fast Fourier transform (FFT) on each of the eigenvectors (eᵢ: i=1, . . . , M) in the eigenvector matrix (E)

The kurtosis analyzer 530 may extract or compute kurtosis or normalized kurtosis of each eigenvector (eᵢ). In more detail, the kurtosis analyzer 530 restores a plurality of two-dimensional (2-D) data matrices (Xᵢ: i=1, . . . , M), each two-dimensional (2-D) data matrix (Xᵢ) corresponding to each eigenvector (eᵢ), according to Equation (2) below.

Xᵢ=eᵢ(eᵢ^{T}eᵢ)⁻¹eᵢeᵢ^{T}X (2)

Also, the kurtosis analyzer 530 may rebuild each two-dimensional (2-D) data matrix (Xᵢ) as a one-dimensional data and extract the kurtosis of each eigenvector (eᵢ). Here, the kurtosis computed to be zero (0) may be regarded as a Gaussian distribution.

The second noise remover 550 may remove the second noise (e.g., the helium pump noise or the cryogenic pump noise) in the EEG signal (X). The EEG signal (X) may be the two-dimensional (2-D) data matrix (X). The second noise remover 550 may remove the second noise based on at least one of the center-frequency (f_{c}) and the kurtosis.

When removing the second noise based on the center-frequency (f_{c}), the eigenvector, among the eigenvectors (eᵢ: i=1, . . . , M), having the center-frequency (f_{c}) higher than or above a first threshold may be determined as a component composing the second noise.

When removing the second noise based on the kurtosis, the eigenvector corresponding to a two-dimensional (2-D) matrix, among the two-dimensional (2-D) matrices (Xᵢ: i=1, . . . , M), having the kurtosis lower than or below a second threshold may be determined as a component composing the second noise. For example, the second threshold may be −0.5 and a distribution of the kurtosis below the second threshold may be a sub-Gaussian distribution.

The second noise remover 550 may generate EEG signal with the second noise removed (\hat{X}) by subtracting a two-dimensional (2-D) data matrix of eigenvectors, which satisfy above conditions, from an original signal (X) according to Equation (3) below.

\hat{X}=X−Σₑ_{ᵣ}_{∈S}_{_{R}}eᵣeᵣ^{T}X^{(r)} (3)

As such, the second noise remover 550 may generate a one-dimensional EEG signal with the second noise removed by restoring the EEG signal with the second noise removed (\hat{X}) as a one-dimensional signal. Restoring as the one-dimensional EEG signal may be in a reverse order of generating a two-dimensional data matrix, and detailed description is thus omitted.

According to an embodiment, the second noise remover 550 may provide a user with an index or pointer for removing the second noise, with a given output device. Also, the second noise remover 550 may remove the second noise in the EEG signal (X) in response to input from the user.

FIG. 4 shows the eigenvectors generated by the data matrix generation module shown in FIG. 1 and the center-frequency for each of the eigenvectors determined by the Fourier transformer shown in FIG. 3.

Referring to FIG. 1, FIG. 3, and FIG. 4, when the size (M) of the segment is, for example, 220, the data matrix generation module 300 may generate 220 number of the eigenvectors (eᵢ: i=1, . . . , M). When the size(M) is 220, the center frequency (f_{c}) for each of the eigenvectors (eᵢ: i=1, . . . , M) determined by the Fourier transformer 510 are as shown in FIG. 4. For example, the center-frequency (f_{c}) for a first eigenvector (e₁) is 8.16 Hz, the center-frequency (f_{c}) for a second eigenvector (e₂) is 15.47 Hz, . . . for a 219th eigenvector (e₂₁₉), 42.97 Hz, and the center-frequencies (f_{c}) for a 220th eigenvector (e₂₂₀) are 45.33 Hz and 11.17 Hz.

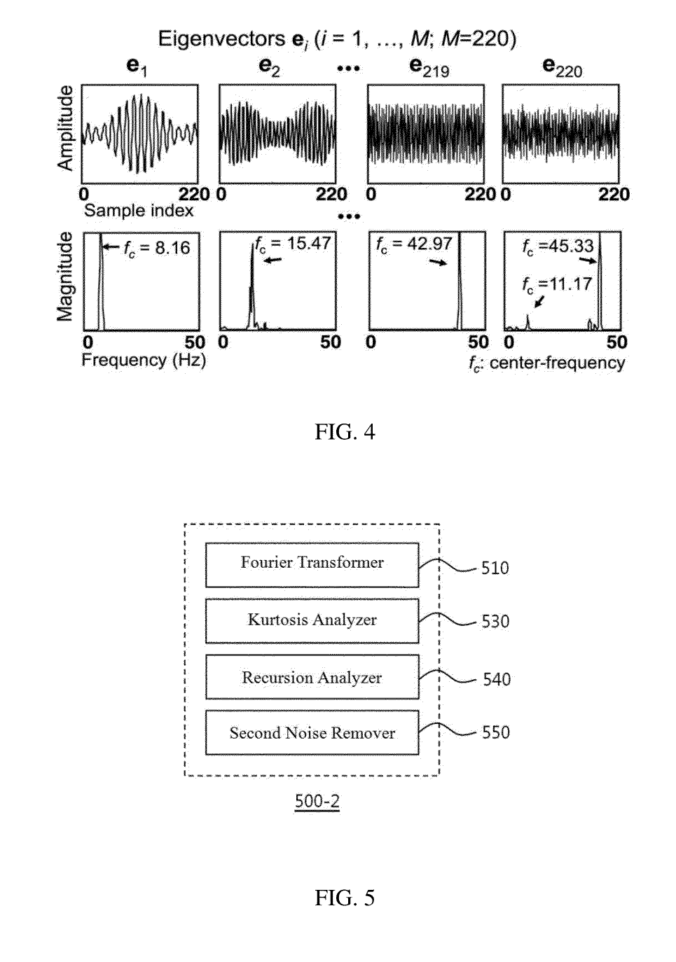

FIG. 5 shows another embodiment of the second noise removal module shown in FIG. 1.

Referring to FIG. 1 and FIG. 5, the second noise removal module 500-2 comprises the Fourier transformer 510, the kurtosis analyzer 530, a recursion analyzer 540, and the second noise remover 550.

Detailed description as to functional and operational elements that are analogous or shared by the second noise removal modules 500-1 (above) and 500-2 (below) are omitted.

The recursion analyzer 540 may analyze and separate an eigenvector having multiple peaks as eigenvectors having a single peak. Here, the eigenvector having the multiple peaks may be deemed to be an eigenvector having another (e.g., more than one, different) peak with peak amplitude higher than or exceeding a third threshold relative to the maximum peak amplitude in the frequency domain. The third threshold may be 1%. That is, an eigenvector having peak amplitude of more than or above 1% of the maximum peak amplitude may be the eigenvector having the multiple peaks.

In more detail, the recursion analyzer 540 may generate at least two eigenvectors from a two-dimensional (2-D) data matrix (Xᵢ) corresponding to the eigenvector having the multiple peaks. Generating the at least two eigenvectors may be analogous to that of eigenvectors or an eigenvector matrix(E) by the PCA module 400 shown in FIG. 1, and detailed description is thus omitted. Here, the size (M) of the segment may be equal to a number of the at least two eigenvectors generated. That is, when separating an eigenvector having two multiple peaks as two eigenvectors, the size (M) may be 2, and when separating an eigenvector having three multiple peaks as three eigenvectors, the size (M) may be 3.

The Fourier transformer 510 may determine the center-frequency (f_{c}) for the eigenvectors additionally generated by the recursion analyzer 540.

The kurtosis analyzer 530 may extract or compute the kurtosis or the normalized kurtosis of each eigenvector (eᵢ) generated by the PCA module 400, as well as those for the eigenvectors additionally generated by the recursion analyzer 540.

The second noise remover 550 may remove the second noise (e.g., the helium pump noise or the cryogenic pump noise) in the EEG signal (X). The second noise remover 550 may remove the second noise based on at least one of the center-frequency (f_{c}), the kurtosis, and data related to the multiple peaks (e.g., existence thereof). That is, when removing the second noise, of the eigenvector having a single peak, the eigenvectors meeting center-frequency and kurtosis conditions may be determined as a component composing the second noise.

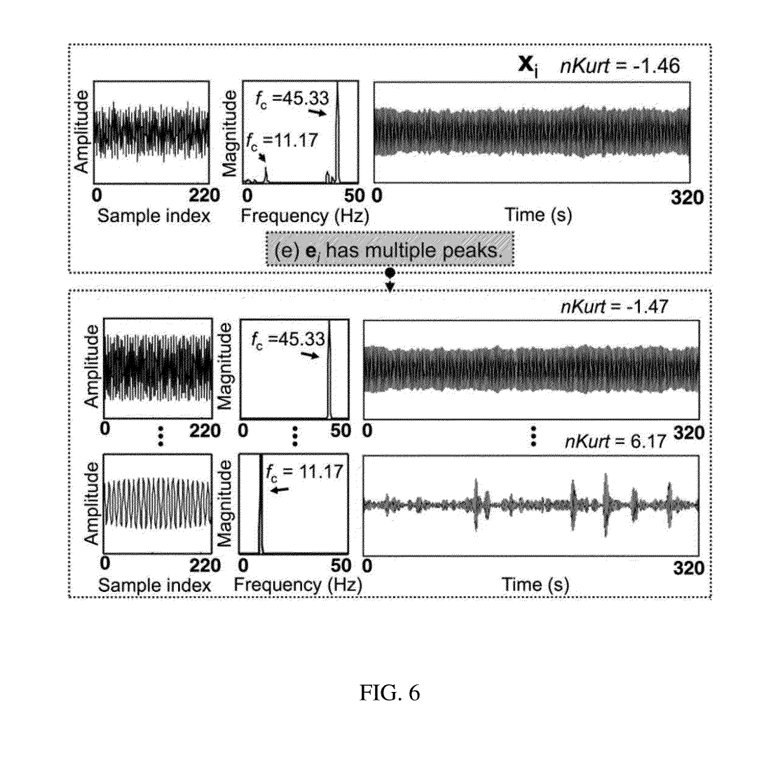

FIG. 6 shows an eigenvector having the multiple peaks as determined and two eigenvectors as separated by the recursion analyzer shown in FIG. 5.

Referring to FIG. 5 and FIG. 6, the 220th eigenvector (e₂₂₀) is analyzed and determined to have the multiple peaks. The recursion analyzer 540 may separate the 220th eigenvector (e₂₂₀) as a 220-1st eigenvector (e₂₂₀₋₁) having the center-frequency (f_{c}) of 45.33 Hz and the kurtosis of −1.47 and a 220-2nd eigenvector (e₂₂₀₋₂) having the center-frequency (f_{c}) of 11.17 Hz and the kurtosis of 6.17.

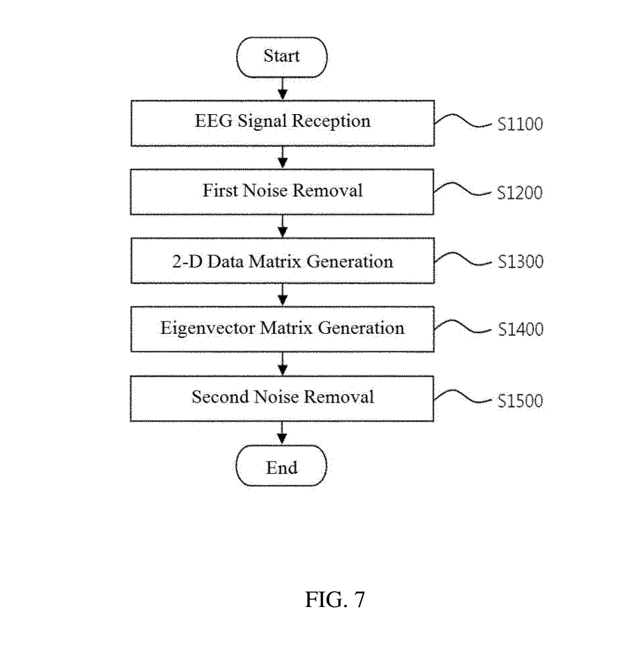

FIG. 7 shows a flow chart of a method for denoising of EEG signal, using the device for denoising of EEG signal 10 shown in FIG. 1.

Referring to FIG. 1 and FIG. 7, the method for denoising of EEG signal, using the device 10, is described in detail, below.

In S1100, EEG signal from an EEG measurement device is received by the signal reception module 100 in the device 10. The EEG signal may be a plurality of signals, with each of the signals from each of multiple channels, or a signal from a single or one particular channel. The EEG signal may be a signal with or without a first noise removed.

In S1200, the first noise in the EEG signal received by the signal reception module 100 may be removed by the first noise removal module 200 in the device 10. The first noise may include at least one of MR gradient artifact/noise, electrocardiography noise, and ballistocardiogram noise.

In S1300, a two-dimensional (2-D) data matrix (X) may be generated by the data matrix generation module 300 in the device 10 from the EEG signal with the first noise removed by the first noise removal module 200 or from the EEG signal received by the signal reception module 100. The two-dimensional (2-D) data matrix (X) is generated by the data matrix generation module 300 from a one-dimensional EEG signal (x) detected or measured from one channel.

In S1400, eigenvectors or an eigenvector matrix (E) may be generated by the PCA module 400 from the two-dimensional (2-D) data matrix(X), using the PCA.

In S1500, a second noise in the EEG signal may be removed by the second noise removal module 500 in the device 10. The second noise may be helium pump noise or cryogenic pump noise. (Method of second noise removal (in the second noise removal module 500) was described in detail earlier, referring to FIG. 3 and FIG. 4.)

The device and method for denoising of EEG signal, according to embodiments of the present invention, may effectively remove noise—among others, helium pump noise and cryogenic pump noise, which are generated in each EEG-signal channels. Further, noise may be removed with minimal EEG-signal loss.

The foregoing description concerns exemplary embodiments of the present invention, which are intended to be illustrative, and should not be construed as limiting the present invention. Many modifications and variations may be made without departing from the spirit and scope of the present invention, as will be readily apparent to persons skilled in the art and as claimed below.

1. A method for denoising of electroencephalography (EEG) signal, comprising:
generating a two-dimensional data matrix (X) from a one-dimensional EEG signal (x), based on segmentation;
generating an eigenvector matrix (E) from the two-dimensional data matrix (X), using principal component analysis (PCA); and
removing noise in the one-dimensional EEG signal (x), based on a center-frequency and kurtosis for each of a plurality of eigenvectors.

2. The method for denoising of EEG signal according to claim 1, wherein the one-dimensional EEG signal (x) is detected base on concurrent EEG-fMRI (functional magnetic resonance imaging) technique.

3. The method for denoising of EEG signal according to claim 1, wherein the noise is helium pump noise or cryogenic pump noise.

4. The method for denoising of EEG signal according to claim 1, wherein the generating the two-dimensional data matrix (X) comprises:
segmenting the one-dimensional EEG signal (x) into a plurality of segments, and
generating the two-dimensional data matrix (X) having as a column component, data in each of the plurality of the segments.

5. The method for denoising of EEG signal according to claim 1, wherein the generating the eigenvector matrix (E) comprises
generating a covariance matrix of the two-dimensional data matrix (X),
wherein the covariance matrix is used as input data for the PCA.

6. The method for denoising of EEG signal according to claim 1, wherein the removing the noise comprises
identifying noise components using the eigenvectors,
wherein an eigenvector having a center-frequency which is greater than or equal to a first threshold and kurtosis which is less than or equal to a second threshold is identified as one of the noise components.

7. The method for denoising of EEG signal according to claim 1, further comprising:
separating an eigenvector having multiple peaks into at least two or more eigenvectors with single peak, after the generating the eigenvector matrix (E).

8. The method for denoising of EEG signal according to claim 7, wherein the eigenvector having the multiple peaks is
an eigenvector whose amplitude of a second peak is above a third threshold in the frequency domain,
wherein the third threshold is predetermined via a percentage of the maximum peak amplitude of the corresponding eigenvector in a frequency domain.

9. A device for denoising of electroencephalography (EEG) signal, comprising:
a data matrix generation module for generating a two-dimensional data matrix (X) from a one-dimensional EEG signal (x), based on segmentation;
a principal component analysis (PCA) module for generating an eigenvector matrix (E) from the two-dimensional data matrix (X), using PCA; and
a noise removal module for removing noise in the one-dimensional EEG signal (x), based on a center-frequency and kurtosis for each of a plurality of eigenvectors.
